(19) United States
(12) Patent Application Publication
(10) Pub. No.: US 2024/0019413 A1
(43) Pub. Date: Jan. 18, 2024

(54) MANUFACTURING METHOD OF OPTICAL FIBER CHEMICAL RATIOMETRIC SENSOR MEASUREMENT SYSTEM FOR MEASURING UNDERWATER DISSOLVED OXYGEN CONCENTRATION

(71) Applicant: TIANJIN UNIVERSITY, Tianjin (CN)

(72) Inventors: Hongxia ZHANG, Tianjin (CN); Yongkun ZHAO, Tianjin (CN); Qingyang MENG, Tianjin (CN); Dagong JIA, Tianjin (CN); Tiegen LIU, Tianjin (CN)

(21) Appl. No.: 18/217,824

(22) Filed: Jul. 3, 2023

(30) Foreign Application Priority Data
Jul. 14, 2022 (CN) 202210824313.3

(51) Int. Cl. G01N 33/18

(52) U.S. Cl. CPC G01N 33/1806

(57) ABSTRACT

The present disclosure discloses a manufacturing method of an optical fiber chemical ratiometric sensor measurement system for measuring underwater dissolved oxygen concentration. The manufacturing method includes the steps of firstly, preparing a carrier substrate to obtain a solution A; then, preparing an oxygen sensitive dye to obtain a solution B; preparing a reference dye to obtain a solution C; mixing the solution A, the solution B and the solution C according to a ratio of 2:1:1 to obtain a composite sensitive dye; depositing the composite sensitive dye on one end face of a sensing optical fiber to prepare a fiber optic probe; establishing the optical fiber chemical ratiometric sensor measurement system, receiving the optical signal by a spectrometer as a fluorescence spectrum, and finally saving and processing fluorescence spectral data by computer software.

### CROSS-REFERENCE TO RELATED APPLICATION

This application claims priority from 202210824313.3 filed Jul. 14, 2022, the content of which is incorporated herein in the entirety by reference.

### TECHNICAL FIELD

The present disclosure belongs to the field of optical measurement, and particularly relates to a manufacturing method of an optical fiber chemical ratiometric sensor measurement system for measuring underwater dissolved oxygen concentration.

### BACKGROUND

Optical dissolved oxygen measurement sensors are classified into two categories: an optical dissolved oxygen measurement sensor based on luminous intensity and an optical dissolved oxygen measurement sensor based on fluorescent lifetime according to measurement methods. The measurement method based on the fluorescent lifetime is not affected by a light source. However, there are relatively fewer sensitive dyes, and the fluorescent lifetime may be changed. The optical chemical dissolved oxygen sensor is generally prepared based on a collisional quenching principle of oxygen molecules and fluorescent or phosphorescent molecules in a carrier substrate. Compared with electrochemical sensors prepared based on a Winkler iodometry method and a Clark electrode method, the optical chemical dissolved oxygen sensor has the advantages of no oxygen consumption, simple operation, low cost, anti-electromagnetic interference and the like. A design method of a ratiometric sensor serves as a compromise, and thus the optical dissolved oxygen sensors based on two different luminophors show different performances at different oxygen concentrations to reduce influences of nonanalytes on change in a light intensity and establish a more robust sensor system.

A Chinese patent CN216284936U disclosed a portable sensor for detecting dissolved oxygen in water by a fluorescence method, which applies a fluorescence quenching principle, and influences of an external light intensity and other interference factors on instrument measurement are avoided. A Chinese patent CN112147121A disclosed a dissolved oxygen monitoring system based on a hydrogel optical fiber, which is used for monitoring a dissolved oxygen concentration and is highly biocompatible with tissues, and thus severe rejection response of the tissues can be avoided. All sensors in the above patents are prepared based on the fluorescence quenching principle, the dissolved oxygen concentration is measured by measuring a fluorescence intensity. However, influences caused by uneven indicator distribution, indicator attenuation and light source fluctuation are not considered.

### SUMMARY

The present disclosure aims to provide a manufacturing method of a ratiometric fiber optic chemical sensor system for measuring a concentration of dissolved oxygen in water. By applying a process of depositing a composite sensitive dye on an end face of a fiber optic probe, the manufacture of the ratiometric fiber optic chemical sensor system for measuring the dissolved oxygen concentration in water is achieved.

The present disclosure provides a manufacturing method of an optical fiber chemical ratiometric sensor measurement system for measuring underwater dissolved oxygen concentration, specifically including the following steps:
step 1, mixing ethyl silicate and n-octyltriethoxysilane to prepare a precursor solution, adding guaranteed reagent absolute ethyl alcohol and hydrogen chloride with a concentration of 0.1 M to a sol solution to catalyze a hydrolysis reaction of organically modified silicate, sealing it by a cover, and magnetically stirring for 1 hour; adding Triton X-100 during mixing to improve a homogeneity of a silica sol, forming a crack-free whole, and preparing a carrier substrate according to a ratio of the ethyl silicate to the n-octyltriethoxysilane to the absolute ethyl alcohol to the 0.1 M hydrogen chloride to the Triton X-100, which is 40:2:12.5:4:1, to obtain a solution A;
step 2, dissolving 4 mg of tri(4,7-biphenyl-1,10-phenanthroline) ruthenium dichloride (II) complex with a purity of 98% in 10 mL of absolute ethyl alcohol to prepare an oxygen sensitive dye, to obtain a solution B;
step 3, dissolving 2 mg of 7-amino-4-trifluoromethyl coumarin in 5 mL of absolute ethyl alcohol and 5 mL of 3-isocyanopropyl-triethoxysilane to prepare a reference dye, to obtain a solution C;
step 4, mixing the solution A, the solution B and the solution C according to a ratio of 2:1:1, to obtain a composite sensitive dye;
step 5, performing dip-coating operation by an HT-DC300 dip coater at 15 mm/min, depositing the composite sensitive dye on one end of an FC/PC-BGUV1000/1100-0.6 sensing optical fiber, and finally, stably holding the sensing optical fiber properly coated in a dry room temperature environment for a week to prepare a fiber optic probe; and
step 6, establishing the optical fiber chemical ratiometric sensor measurement system for measuring the concentration of the dissolved oxygen, taking an LD light source with a central wavelength of 405 nm as an excitation light source, entering one end of the sensing optical fiber to an oxygen sensitive membrane of the fiber optic probe, transmitting an optical signal through the other end of the sensing optical fiber after fluorescence excitation, receiving the optical signal as a fluorescence spectrum, and finally saving and processing spectral data.

Compared with the prior art, the present disclosure achieves measurement of the dissolved oxygen concentration, improves the stability of a sensor, and reduces influences caused by light source fluctuation, indicator attenuation and the like.

### BRIEF DESCRIPTION OF THE DRAWINGS

FIG. 1 is a schematic diagram of a manufacturing method of an optical fiber chemical ratiometric sensor measurement system for measuring underwater dissolved oxygen concentration according to the present disclosure.

FIG. 2 is a structural schematic diagram of an optical fiber chemical ratiometric sensor measurement system for measuring underwater dissolved oxygen concentration according to the present disclosure.

FIG. 3 is a structural schematic diagram of a fiber optic probe 3.

FIGS. 4A-4B are schematic diagrams of experimental results of measurement of underwater dissolved oxygen concentration according to the present disclosure, wherein FIG. 4A is a fluorescence emission spectrum of a sensor at different dissolved oxygen concentrations, and FIG. 4B is a curve graph of a Stern-Volmer equation at different dissolved oxygen concentrations.

FIG. 5 is a curve graph of response time of an optical fiber chemical ratiometric sensor for measuring a concentration of dissolved oxygen in water according to an embodiment of the present disclosure.

FIG. 6 is a diagram illustrating results of a light stability experiment of a optical fiber chemical ratiometric sensor measurement system for measuring underwater dissolved oxygen concentration according to an embodiment of the present disclosure.

FIG. 7 is a diagram illustrating results of a repeated experiment of a ratiometric sensor measurement system for measuring underwater dissolved oxygen concentration according to an embodiment of the present disclosure.

### REFERENCE SIGNS

1. Light source module, 2. Y-type sensing optical fiber, 3. Fiber optic probe, 4. Dissolved oxygen meter, 5. Mixing chamber, 6. Flow controller, 7. Oxygen cylinder, 8. Nitrogen cylinder, 9. Spectrometer, 10. Computer, 11. Sample, 12. Sample cell, 13. Fiber optic connector, 21. Excitation light signal, 22. Emitted light signal, 23. Oxygen sensitive material, 24. Reference material, 61. first flow controller, and 62. second flow controller.

### DETAILED DESCRIPTION OF THE PRESENT DISCLOSURE

The present disclosure will be further described in detail with reference to the drawings and embodiments.

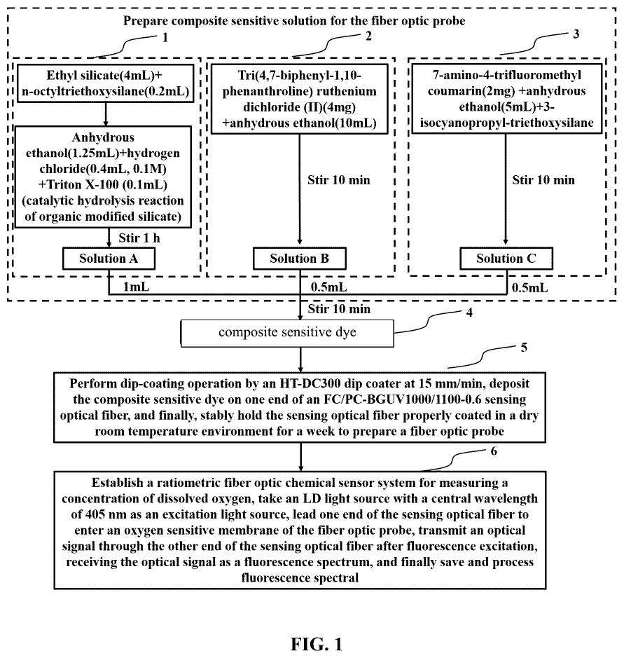

As shown in FIG. 1, it is a diagram of a whole process of a manufacturing method of an optical fiber chemical ratiometric sensor measurement system for measuring underwater dissolved oxygen concentration according to the present disclosure. The manufacturing method specifically includes the following steps:
firstly, a composite sensitive solution of a probe of an optical fiber chemical ratiometric sensor measurement system for measuring underwater dissolved oxygen concentration is prepared specifically as follows:
step 1, ethyl silicate and n-octyltriethoxysilane are mixed to prepare a precursor solution, guaranteed reagent absolute ethyl alcohol and hydrogen chloride with a concentration of 0.1 M are added to a sol solution to catalyze a hydrolysis reaction of organically modified silicate, the sol solution is sealed by a cover, and is magnetically stirred for 1 hour; Triton X-100 is added during mixing to improve a homogeneity of a silica sol, and a crack-free whole is formed, and a carrier substrate is prepared according to a ratio of the ethyl silicate to the n-octyltriethoxysilane to the absolute ethyl alcohol to the 0.1 M hydrogen chloride to the Triton X-100, which is 40:2:12.5:4:1, to obtain a solution A;
step 2, 4 mg of tri(4,7-biphenyl-1,10-phenanthroline) ruthenium dichloride (II) complex with a purity of 98% is dissolved in 10 mL of absolute ethyl alcohol to prepare an oxygen sensitive dye, and a solution B is obtained;
step 3, 2 mg of 7-amino-4-trifluoromethyl coumarin is dissolved in 5 mL of absolute ethyl alcohol and 5 mL of 3-isocyanopropyl-triethoxysilane to prepare a reference dye, and a solution C is obtained;
step 4, the solution A, the solution B and the solution C are mixed according to a ratio of 2:1:1 to obtain a composite sensitive dye;
step 5, dip-coating operation is performed by an HT-DC300 dip coater at 15 mm/min, the composite sensitive dye is deposited on one end of an FC/PC-BGUV1000/1100-0.6 sensing optical fiber, and finally, the sensing optical fiber properly coated is stably held in a dry room temperature environment for a week to prepare a fiber optic probe; and
step 6, the optical fiber chemical ratiometric sensor measurement system for measuring underwater dissolved oxygen concentration is established, an LD light source with a central wavelength of 405 nm serves as an excitation light source, one end of a Y-type sensing optical fiber is entered to an oxygen sensitive membrane of the fiber optic probe, an optical signal is transmitted through the other end of the Y-type sensing optical fiber after fluorescence excitation and received by a spectrometer, and finally spectral data is saved and processed by computer software.

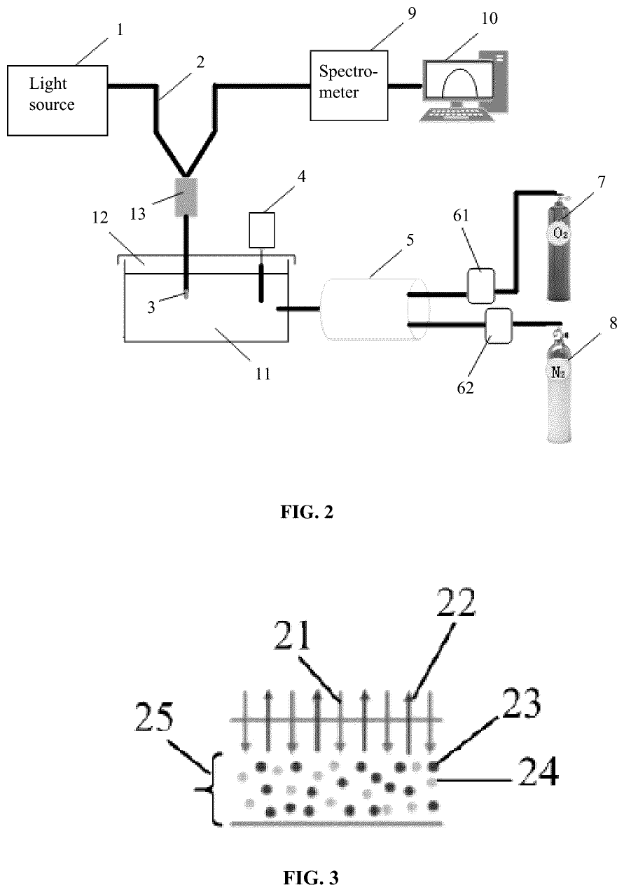

The embodiment of the present disclosure is described as follows:
step 1, an optical fiber chemical ratiometric sensor measurement system for measuring underwater dissolved oxygen concentration is established, FIG. 2 shows a structural schematic diagram of the optical fiber chemical ratiometric sensor measurement system for measuring underwater dissolved oxygen concentration according to the present disclosure. The system includes a light source module 1, a Y-type sensing optical fiber 2, a fiber optic probe 3, a dissolved oxygen meter 4, a mixing chamber 5, a first flow controller 61, a second flow controller 62, an oxygen cylinder 7, a nitrogen cylinder 8, a spectrometer 9, and a computer 10. Wherein, the light source module 1 is an MW-GX-405/150 mW LD type light source, and an excitation light signal output from the light source module 1 enters the fiber optic probe 3 through one end of the Y-type sensing optical fiber 2; nitrogen in the nitrogen cylinder 8 is filled into the mixing chamber 5 through the second flow controller 62 at a certain flow rate for 30 minutes, thereby enabling the mixing chamber 5 to meet the standard of an oxygen-free environment to prepare a saturated nitrogen solution; a first fluorescence excitation phenomenon of the dissolved oxygen meter 4 in the saturated nitrogen solution for soaking a sample 11 is observed, and then oxygen in the oxygen cylinder 7 is filled into the mixing chamber 5 at a certain flow rate through the first flow controller 61 at intervals of 5 to 30 seconds; the fluorescence spectrum changes of an oxygen sensor are observed, and fluorescence spectra of sample solutions with different dissolved oxygen concentrations are acquired and calculated by the dissolved oxygen meter 4; a light signal is emitted through the other end of the Y-type sensing optical fiber 2 after fluorescence excitation, and the spectrometer 9 receives the emitted light signal; and finally the fluorescence spectral data is saved and processed by the computer 10.

FIG. 3 shows a structural schematic diagram of the fiber optic probe 3. The fiber optic probe 3 includes an oxygen sensitive membrane consisting of an oxygen sensitive material 23 and a reference dye 24. The excitation light signal 21 serves as an input, and the emitted light signal 22 serves as an output.

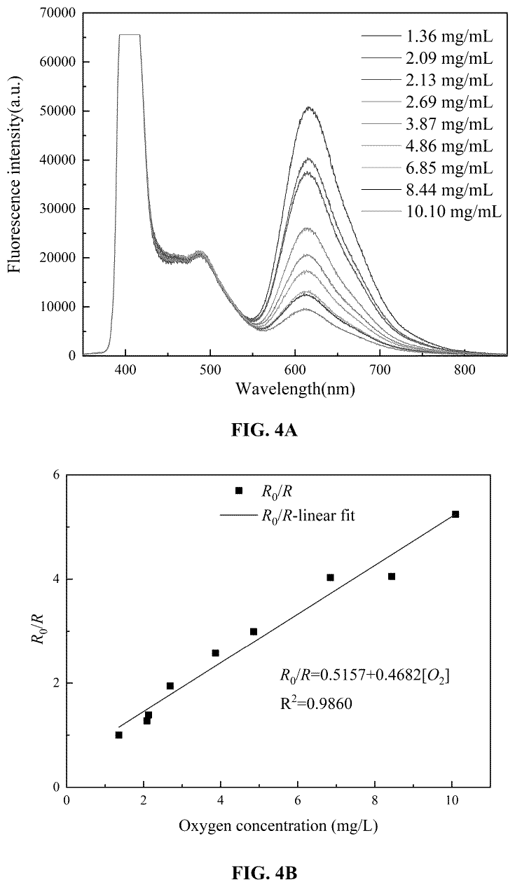

Step 2, the fluorescence spectra of the sample solutions with different dissolved oxygen concentrations in water are acquired and calculated by the dissolved oxygen sensor:
firstly, a flask with two necks containing a certain volume of deionized water is heated to boil the deionized water for 10 minutes, and then sealed and cooled to a room temperature; next, the nitrogen is filled into the flask at a certain flow rate for 30 minutes, thereby enabling the flask to meet the standard of the oxygen-free environment to prepare the saturated nitrogen solution, and the first fluorescence excitation phenomenon of the oxygen sensor in the saturated nitrogen solution is observed; then, oxygen is filled into the flask at a certain flow rate every 5 to 30 seconds (it can be known from experiments that the relationship between the solubility of the oxygen in water is not in an absolutely linear relation with time, the same flow rate of oxygen is dissolved quickly at the beginning, and the longer the duration, the lower the rate of dissolution); and the fluorescence spectrum changes of the oxygen sensor are observed in sequence. FIGS. 4A-4B shows a schematic diagram of experimental results of measurement of dissolved oxygen concentration by the optical fiber chemical ratiometric sensor measurement system according to the embodiment of the present disclosure; wherein FIG. 4A is a fluorescence emission spectrum of a sensor at different dissolved oxygen concentrations. After excitation by the LD light source, AFC and a ruthenium complex in an aqueous solution have different spectral responses at different oxygen concentrations. The AFC and the ruthenium complex have two emission peaks at 490 nm and 615 nm or so respectively, and a peak value represents a fluorescence intensity; it can be seen that the fluorescence intensity of the ruthenium complex gradually decreases due to a fluorescence quenching effect with an increase in the oxygen concentration, while the fluorescence intensity of the AFC is stabilized at a certain value.

The optical dissolved oxygen measurement sensor generally uses a fluorescent substance as an indicator to generate fluorescence under excitation of light with a specific wavelength; and the oxygen has a quenching effect on the fluorescence, resulting in a decrease in the fluorescence intensity, and the purpose of detecting the oxygen concentration is achieved by detecting the quenching degree of the indicator. The relationship between the fluorescence intensity and the oxygen concentration is allowed to be described with a Stern-Volmer equation:

$$\frac{{I}_{0}}{I} = 1 + {K}_{SV} [ {O}_{2} ] \qquad ( 1 )$$

Where, I₀ and I represent fluorescence intensities under anaerobic and aerobic conditions respectively, K_{SV} is a Stern-Volmer quenching constant, and [O₂] represents an oxygen concentration.

Although the dissolved oxygen concentration is allowed to be detected only by using the ruthenium complex, once the light source fluctuates greatly, the fluorescence intensity of the ruthenium complex will be changed greatly, and accordingly the fluorescence intensity is inconsistent with that previously calibrated, leading to inaccurate measurement. Thus, a ratiometric method is used for detection of the oxygen sensor, and the Stern-Volmer equation is simply promoted, as shown in the following formula:

$$\frac{{R}_{0}}{R} = \frac{{I}_{0} / {I}_{AFC 0}}{I / {I}_{AFC}} = 1 + {K}_{SV} [ {O}_{2} ] \qquad ( 2 )$$

Where, I_{AFCO} and I_{AFC} represent the fluorescence intensities of coumarin under the anaerobic and aerobic conditions respectively, and R₀ and R represent the ratios of the fluorescence intensities of the ruthenium complex to those of the coumarin under the anaerobic and aerobic conditions respectively.

FIG. 4B is a Stern-Volmer equation curve at different dissolved oxygen concentrations. The fluorescence intensity of the AFC at 490 nm and the fluorescence intensity of the ruthenium complex at 615 nm are acquired respectively, and the Stern-Volmer equation curve simply promoted is plotted according to the formula (2). The relative fluorescence intensity of the dissolved oxygen sensor has a good linear relationship with the dissolved oxygen concentration, which indicates that almost all ruthenium complex molecules are in the same microenvironment, the linearity is up to 98.60%, and the sensitivity is up to 0.4682/unit [O₂].

Step 3, the response time, stability and repeatability of the sensor are characterized.

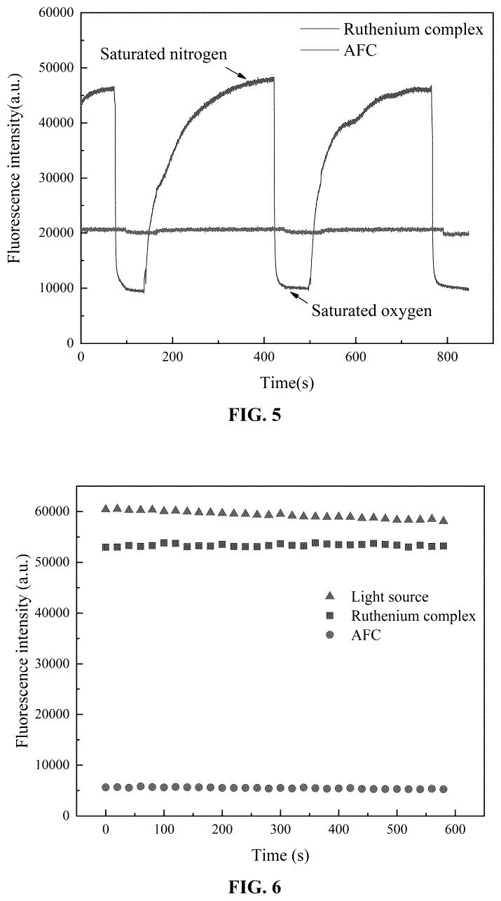

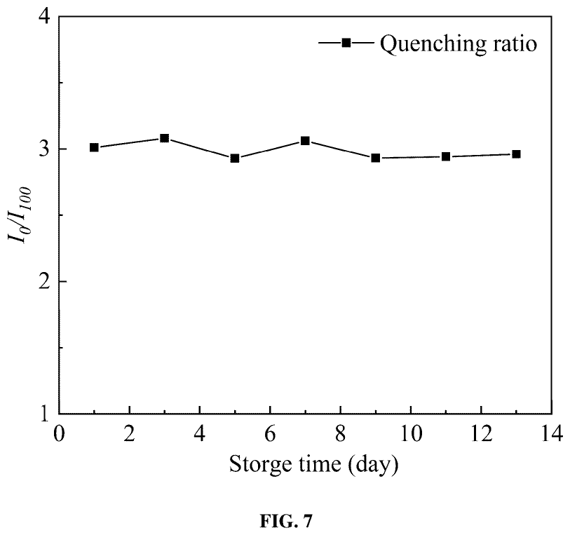

FIG. 5 shows a curve graph of response time of an optical fiber chemical ratiometric sensor measurement system according to the embodiment of the present disclosure. The response time refers to the time required for a 90% change in an equilibrium value intensity reading. It can be seen from the figure that the fluorescence intensities of the ruthenium complex and the AFC are kept basically unchanged either in the saturated nitrogen solution or in the saturated oxygen solution. The response time from the saturated nitrogen solution to the saturated oxygen solution is 12 seconds, and the response time from the saturated oxygen solution to the saturated nitrogen solution is 144 seconds, which shows a good response characteristic. As shown in FIG. 6, it is a diagram illustrating results of a light stability experiment of the optical fiber chemical ratiometric sensor measurement system according to the embodiment of the present disclosure. The power of the LD light source is kept unchanged, fluorescence emission conditions of the ruthenium complex and the AFC in the saturated nitrogen solution within a certain period are observed, data is acquired every 20 seconds, and the fluctuation values of the fluorescence intensities of the ruthenium complex and the AFC are analyzed. Standard deviations of the fluorescence intensities of the ruthenium complex and the AFC under the LD light source and the saturated nitrogen solution are calculated, which are 713.08, 250.49 and 155.33 respectively, and are relatively stable compared with a full scale range. As shown in FIG. 7, it is a diagram illustrating results of a repeated experiment of the optical fiber chemical ratiometric sensor according to the embodiment of the present disclosure. In order to detect the repeatability of the prepared optical fiber chemical ratiometric sensor, on the premise of ensuring consistent test conditions, the same sensing optical fiber is subjected to fluorescence excitation by using the light source with the same power, and the experimental interval periods are 1 day, 3 days, 5 days, 7 days, 9 days, 11 days and 13 days respectively; the fluorescence quenching conditions of the sensing optical fiber in the saturated nitrogen solution and the saturated oxygen solution are observed, and a quenching ratio is calculated; and a total of 7 sets of experimental data are acquired, the quenching ratio is stabilized at 3 or so, and the repeatability is higher.

The above descriptions are merely the preferred specific implementations of the present disclosure, which are not intended to limit the protection scope of the present disclosure. Any change or replacement, which was easily conceived by any person skilled in the art according to the manufacturing method under the process conditions of zoom-in or zoom-out at the same proportion, within the technical scope disclosed in the present disclosure shall fall within the protection scope of the present disclosure. Thus, the protection scope of the present disclosure should be subject to the protection scope of the claims.

What is claimed is:

1. A manufacturing method of an optical fiber chemical ratiometric sensor measurement system for measuring underwater dissolved oxygen concentration, comprising the following steps:
step 1, mixing ethyl silicate and n-octyltriethoxysilane to prepare a precursor solution, adding guaranteed reagent absolute ethyl alcohol and hydrogen chloride with a concentration of 0.1 M to a sol solution to catalyze a hydrolysis reaction of organically modified silicate, sealing it by a cover, and magnetically stirring for 1 hour; adding Triton X-100 during mixing to improve a homogeneity of a silica sol, forming a crack-free whole, and preparing a carrier substrate according to a ratio of the ethyl silicate to the n-octyltriethoxysilane to the absolute ethyl alcohol to the 0.1 M hydrogen chloride to the Triton X-100, which is 40:2:12.5:4:1, to obtain a solution A;
step 2, dissolving 4 mg of tri(4,7-biphenyl-1,10-phenanthroline) ruthenium dichloride (II) complex with a purity of 98% in 10 mL of absolute ethyl alcohol to prepare an oxygen sensitive dye, to obtain a solution B;
step 3, dissolving 2 mg of 7-amino-4-trifluoromethyl coumarin in 5 mL of absolute ethyl alcohol and 5 mL of 3-isocyanopropyl-triethoxysilane to prepare a reference dye, to obtain a solution C;
step 4, mixing the solution A, the solution B and the solution C according to a ratio of 2:1:1, to obtain a composite sensitive dye;
step 5, performing dip-coating operation by an HT-DC300 dip coater at 15 mm/min, depositing the composite sensitive dye on one end face of an FC/PC-BGUV1000/1100-0.6 sensing optical fiber, and then stably holding the sensing optical fiber properly coated in a dry room temperature environment for a week to prepare a fiber optic probe; and
step 6, establishing the optical fiber chemical ratiometric sensor measurement system for measuring dissolved oxygen concentration, taking an LD light source with a central wavelength of 405 nm as an excitation light source, ertering one end of the sensing optical fiber to an oxygen sensitive membrane of the fiber optic probe, transmitting an optical signal through the other end of the sensing optical fiber after fluorescence excitation, receiving the optical signal as a fluorescence spectrum, and finally saving and processing fluorescence spectral data.

2. The manufacturing method of the optical fiber chemical ratiometric sensor measurement system for measuring underwater dissolved oxygen concentration according to claim 1, wherein the FC/PC-BGUV1000/1100-0.6 sensing optical fiber is selected as the sensing optical fiber.
